(19) United States
(12) Patent Application Publication
(10) Pub. No.: US 2019/0132593 A1
(43) Pub. Date: May 2, 2019

(54) CT DATA PROCESSING

(71) Applicant: SHENYANG NEUSOFT MEDICAL SYSTEMS CO., LTD., Shenyang (CN)

(72) Inventors: Shanshan LOU, Shenyang (CN); Changkun LIU, Shenyang (CN)

(21) Appl. No.: 16/175,693

(22) Filed: Oct. 30, 2018

(30) Foreign Application Priority Data
Oct. 31, 2017 (CN) 201711050549.1

(51) Int. Cl. H04N 19/146; A61B 6/03; A61B 6/06; A61B 6/00

(52) U.S. Cl. CPC H04N 19/146; A61B 6/56; A61B 6/06; A61B 6/032

(57) ABSTRACT

A method and device for processing CT data in a CT system are provided. According to an example of the method, when an upper limit of a data transmission bandwidth of a CT machine is determined, an amount of scan data collected per unit time by the CT machine may be taken as a data acquisition amount per unit time and a first ratio of the data acquisition amount per unit time to the upper limit of the data transmission bandwidth may be determined. In this way, a compression strategy and a decompression strategy may be determined according to the first ratio.

### CROSS REFERENCE TO RELATED APPLICATIONS

The present application claims priority to Chinese Patent Application No. 201711050549.1 entitled “CT DATA PROCESSING,” filed on Oct. 31, 2017. The entire contents of the above-listed application are hereby incorporated by reference in their entirety for all purposes.

### BACKGROUND

CT (Computed Tomography) imaging is one of major imaging methods in modern medical imaging. A basic principle of the CT imaging is that a subject is scanned with highly sensitive detectors depending on differences of X-ray absorption and transmittance among different tissues of the subject. The detectors receive the X-rays transmitted through the subject and convert them into electrical signals. Then the electrical signals are converted into digital signals. With rapid development of medical technologies, the number of detector slices of a CT machine currently used in clinical practice is increasing. For example, a CT machine with 512-slice detectors, and an amount of data collected per unit time in such CT machine is very large.

NEUSOFT MEDICAL SYSTEMS CO., LTD. (NMS), founded in 1998 with its world headquarters in China, is a leading supplier of medical equipment, medical IT solutions, and healthcare services. NMS supplies medical equipment with a wide portfolio, including CT, Magnetic Resonance Imaging (MRI), digital X-ray machine, ultrasound, Positron Emission Tomography (PET), Linear Accelerator (LINAC), and biochemistry analyser. Currently, NMS' products are exported to over 60 countries and regions around the globe, serving more than 5,000 renowned customers. NMS's latest successful developments, such as 128 Multi-Slice CT Scanner System, Superconducting MRI, LINAC, and PET products, have led China to become a global high-end medical equipment producer. As an integrated supplier with extensive experience in large medical equipment, NMS has been committed to the study of avoiding secondary potential harm caused by excessive X-ray irradiation to the subject during the CT scanning process.

### BRIEF DESCRIPTION OF DRAWINGS

FIG. 1A illustrates a system architecture diagram of an example CT machine.

FIG. 1B illustrates a system architecture diagram of part of a scanner in an example CT machine.

FIG. 2 illustrates a flow chart of processing CT data according to a first example of the present disclosure.

FIG. 3A illustrates a flow chart of processing CT data according to a second example of the present disclosure.

FIG. 3B illustrates a flow chart of step S303 in FIG. 3A.

FIG. 4 illustrates a flow chart of processing CT data according to a third example of the present disclosure.

FIG. 5 illustrates a hardware structure of a CT data processing device according to an example of the present disclosure.

FIG. 6 illustrates functional modules of CT data processing control logic according to an example of the present disclosure.

### DETAILED DESCRIPTION

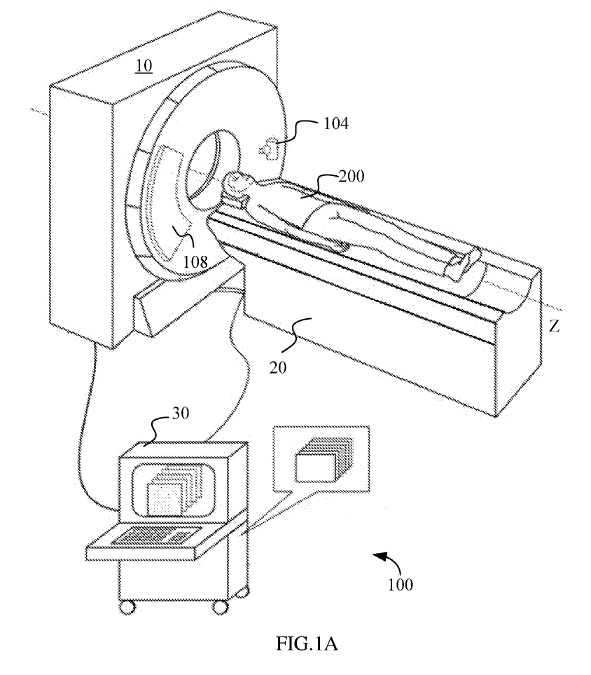

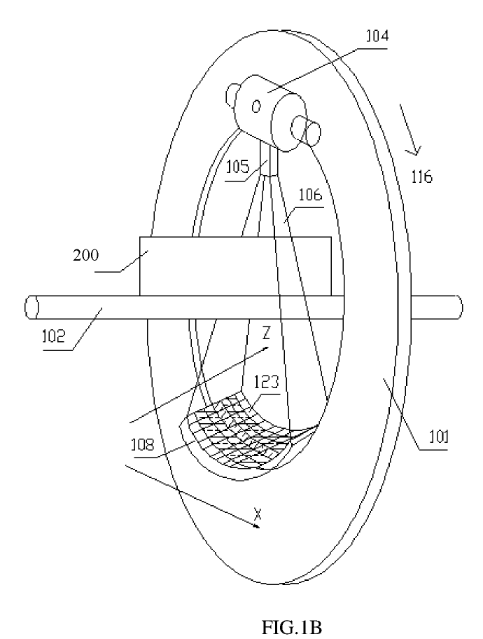

CT machines are widely used in the field of clinical medicine. FIG. 1A shows a system architecture diagram of an example CT machine.

The CT machine 100 generally includes a scanner 10, a scanning bed 20, and a computer system 30.

The scanner 10 is mainly configured to perform scanning with a specific scanning mode to obtain scan data from a scanning slice of a patient 200. The computer system 30 then performs image reconstruction with the scan data to obtain a CT image. A tube 104 in the scanner 10 is configured to project a cone-shaped beam of X-rays, and an array of detectors 108 is mounted in the scanner 10 at a position opposite the tube 104 for detecting intensity of the X-rays.

The scanning bed 20 is configured to accurately move the patient 200 to a predetermined or appropriate location.

The computer system 30 includes three main functions. First, after an operator selects appropriate scanning parameters to initiate a scanning process, the computer system 30 controls the scanner 10 and the scanning bed 20, and schedules various events during the scanning process. Second, a CT image is generated by data processing which includes data pre-processing and image reconstruction. Third, the CT image is displayed in the computer system 30 to the operator.

With rapid development of medical technologies, the number of slices of the detectors in the CT machine currently used in clinical practice is increasing. For example, a CT machine with 512-slice detectors and an amount of scan data collected per unit time in such CT machine is very large. A data acquisition system and a data transmission system are two independent systems located in the scanner. The data acquisition system is used to amplify, integrate, and sample hold weak electrical signals output from detectors, and then mixed into a plurality of paths by a multi-way switch. Finally, digital signals are generated by a plurality of analog-to-digital converters (ADC). The data transmission system is configured to send the digital signals to the computer system for further processing. Due to limitations of hardware performance for data transmission system, transmission bandwidth may be less than the data acquisition rate of the data acquisition system. In this case, the acquisition data needs to be compressed before being sent. Therefore, it is necessary to provide a data processing method to better transmit the CT scanning data.

In the prior art, when the data transmission system transmits data, a fixed compression method is used to compress the acquisition data. This means that a same compression method is adopted regardless of whether the data acquisition amount per unit time is large or small. That is, a same data compression strategy is used for data obtained by different scanning processes. In order to make the compression strategy meet transmission bandwidth requirements for data collected during all scanning scenarios, a data compression strategy with a high compression ratio is generally adopted. It may be understood that when a compression ratio of a compression strategy is large, the transmission takes less time under a certain transmission bandwidth, but data accuracy loss is relatively large. When a compression ratio of a compression strategy is small, data accuracy loss is small, but under the same transmission bandwidth the transmission takes a long time.

When a data acquisition amount per unit time is large, a requirement of the transmission bandwidth is high. At this time, the acquisition data needs to be compressed with a high compression ratio to achieve a fast data transmission rate. When a data acquisition amount per unit time is small, the requirement of the transmission bandwidth is low. A compression strategy with a large compression ratio may result in a large loss of data information and a high computational complexity. According to a method provided by the present disclosure, when the data acquisition amount per unit time is small, the data may be transmitted with a compression strategy having a low compression ratio or even without compression, thereby reducing the data accuracy loss.

### First Example

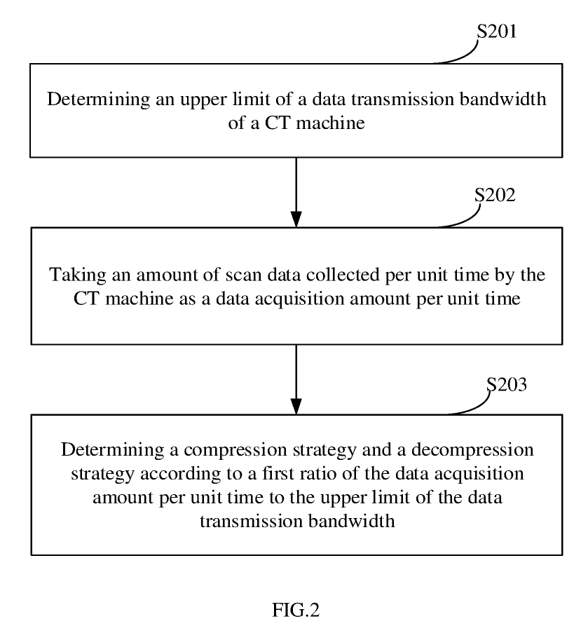

FIG. 2 is a flow chart illustrating a process of processing CT data according to the first example of the present disclosure. The CT data processing method provided in the example may include the following process.

Step S201: an upper limit of a data transmission bandwidth of a CT machine is determined.

The upper limit of the data transmission bandwidth of the CT machine is determined according to hardware performance of the CT machine. The process from data acquisition to data transfer in the CT machine is described as following.

Analog signals acquired by scanning a subject are converted into digital signals through ADCs, and then the digital signals are compressed according to a preset compression strategy. The compressed data are sent to a data receiver through a slip ring and an optical fiber link. The data receiver may select a data decompression strategy corresponding to the data compression strategy to decompression the compressed data.

The upper limit of the data transmission bandwidth of the CT machine is related to a transmission rate of the slip ring and a transmission rate of the optical fiber link.

The transmission rate of the slip ring is set at the slip ring manufactory. Transmission rate specifications of the slip ring may include, for example, 5 Gbps, 10 Gbps, 15 Gbps, and 20 Gbps. Among them, bps represents bits/second; G represents giga, and 5 Gbps represents 5 gigabits/second. Transmission rate specifications of the optical fiber link may include, for example, 5 Gbps, 10 Gbps, 12 Gbps, 15 Gbps, and the like.

It can be understood that the slowest device in the data transmission link of the CT machine determines the upper limit of the data transmission bandwidth of the CT machine. For example, if the transmission rate of the slip ring is 5 Gbps and the transmission rate of the optical fiber link is 10 Gbps, the upper limit of the data transmission bandwidth of the CT machine is 5 Gbps.

Step S202: an amount of scan data collected per unit time by the CT machine is taken as a data acquisition amount per unit time.

The amount of the scan data collected per unit time by the CT machine is related to CT machine parameter values set during scanning. The scan parameter values are set based on dedicated lesions. Different scan parameter values may result different data amounts collected per unit time.

A lesion may be in a head, an abdomen or a chest, etc. Due to the area difference between a small area of a head slice and a large area of an abdomen slice or a chest slice, the corresponding scan parameter values of the CT machine are different, and the amounts of scan data collected per unit time are also different.

For example, the data acquisition amount per unit time corresponding to a lesion in the head may be smaller than that corresponding to a lesion in the abdomen.

In addition, even if lesions are in a same position, differences in patient weight may result in different scan parameter values.

For example, when a lesion scanned by the CT machine is in an abdomen of a patient, an amount of scan data corresponding to the abdomen of an obese patient is different from an amount of scan data corresponding to the abdomen of a thin patient in the same time unit. The data acquisition amount per unit time corresponding to an obese patient may be larger than that corresponding to a thin patient.

Step S203: a compression strategy and a decompression strategy are determined according to a first ratio of the data acquisition amount per unit time to the upper limit of the data transmission bandwidth.

A compression ratio is a ratio of an amount of data before compression to an amount of data after compression. For example, the amount of data before compression is 2 Gb, the amount of data after compression is 1 Gb, and the corresponding compression ratio is 2. Where, Gb represents gigabit. It should be noted that one compression ratio may correspond to one compression strategy, and one compression strategy corresponds to one decompression strategy.

The compression strategy includes lossy compression algorithms and lossless compression algorithms.

The lossy compression algorithms change data itself and round off some information in the data. Generally, the compression ratio of lossy compression is high. That is, lossy compression may be used for scenario that requires a high compression ratio.

The lossless compression algorithms exploit statistical redundancy to represent data without losing any information. For example, a data storage manner is optimized. After compressed data using a lossless compression algorithm is decompressed, all original data information may be restored, and data accuracy may not be changed. Lossless compression may be used for scenario that requires a relative low compression ratio.

For example, when an amount of data before compression is 2 Gb and an amount of data after compression is 1.67 Gb, the compression ratio is 1.2. In this scenario, lossless compression may be adopted, and a decompression strategy is adopted according to the compression strategy.

The first ratio has a certain correspondence with respect to the compression ratio. The correspondence is obtained experimentally according to characteristics of the CT scan data. A range of the first ratios may correspond to a dedicated compression ratio. For example, the upper limit of the bandwidth is 10 Gbps, and the data acquisition amount per unit time is 12 Gb. Here, the unit time is 1 second, that is, the data acquisition rate is 12 Gb/s. The first ratio is 1.2, and the corresponding compression ratio may also be 1.2. For another example, the upper limit of the bandwidth is 10 Gbps. The data acquisition amount per second is 15 Gb. The first ratio is 1.5, and the corresponding compression ratio may be 1.6. It should be noted that the compression ratio corresponding to the compression strategy needs to be greater than or equal to the first ratio, so as to ensure that the compressed data amount per unit time is less than or equal to the upper limit of the data transmission bandwidth.

The CT data processing method provided in the example selects a corresponding compression strategy by using an upper limit of a data transmission bandwidth in the CT machine and an amount of scan data collected per unit time by the CT machine. Different compression strategies are adopted according to different first ratios. In the case that the upper limit of the data transmission bandwidth is determined, the data acquisition amount per unit time is different, and the corresponding compression strategy is also different. Rather than a single compression strategy with a fixed compression ratio in the prior art, the solution provided by the present disclosure may adopt a compression strategy with a large compression ratio for a large amount of data acquired per unit time, and a compression strategy with a small compression ratio or even no compression for a small amount of data acquired per unit time. The greater the compression ratio, the greater data accuracy loss. Therefore, the method provided by the present disclosure may improve the data accuracy as much as possible while ensuring normal data transmission, and fully utilize the bandwidth of a data transmission system.

For the CT data processing method provided by the first example, a second example may further describe an implementation manner of steps S202 and S203.

### Second Example

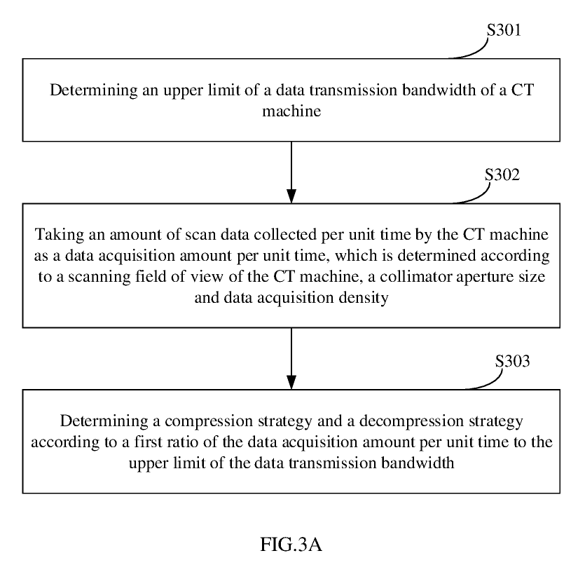

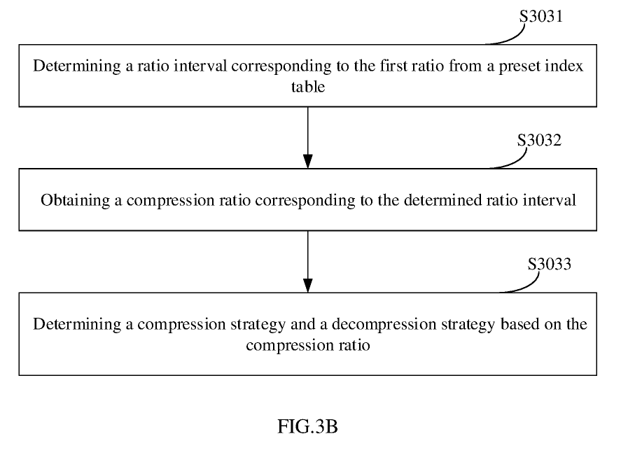

FIG. 3A is a flow chart illustrating a process of processing CT data according to the second example of the present disclosure. The CT data processing method provided in the example includes the following steps.

Step S301: an upper limit of a data transmission bandwidth of a CT machine is determined.

The process of determining the upper limit at step 301 may be similar with that at step 201, which will not be repeated herein.

Step S302: an amount of scan data collected per unit time by the CT machine is taken as a data acquisition amount per unit time, where the data acquisition amount per unit time may be determined according to a scanning field of view of the CT machine, a collimator aperture size and data acquisition density.

From the perspective of the detector, the scanning field of view determines the number of columns of valid detectors. The larger the scanning field of view, the greater the number of columns of the valid detectors. The collimator aperture size determines the number of slices of valid detectors. The larger the collimator aperture size, the greater the number of slices of the valid detectors.

FIG. 1B is a system architecture diagram of part of a scanner in an example CT machine. The CT scanner includes a support frame 101 that is rotatable about a rotational axis 102. The support frame 101 may be driven by a motor. A tube 104 mounted on the support frame 101 is an X-ray source. The tube 104 forms a cone-shaped X-ray beam 106 through an aperture 105. The cone-shaped X-ray beam 106 may pass through a patient 200 placed at the center of a bore of the scanner 10, impinging on an array of detectors 108. The detectors array 108 comprises of a plurality of detectors 123. The detector array 108 is disposed on the support frame 101 at a position opposite the tube 104 such that the surfaces of the detectors 123 may be covered by the cone-shaped X-ray beam 106. During a scan process, the tube 104 and the aperture 105 may rotate with the support frame 101 in a direction indicated by an arrow 116.

The scanning field of view of a CT machine determines the number of columns of the detector array radiated by the X-ray beam when scanning a lesion. That is, the number of columns of the detector array 108 radiated by the X-ray beam 106 in an x-axis direction in FIG. 1B. It can be understood that scan areas related to different lesions are different, and the corresponding scanning field of views are also different. For example, a scan area corresponding to a lesion in an abdomen is different from a scan area corresponding to a lesion in a brain. A scan area corresponding to a lesion in a brain is different from a scan area corresponding to a lesion in a leg. The larger scanning field of view, the larger the number of columns of the valid detectors.

It should be noted that the number of detectors may be very large, but not all detectors participate in the detection during a scanning process. Detectors radiated by the X-ray beam may be taken as valid detectors.

After the scanning field of view and the collimator aperture size are determined, the number of detectors participating in the detection may be determined. Correspondingly, an amount of data output by the valid detectors for one sampling may be determined.

In an example, for a head scanning, a scanning field of view is 250 mm, the corresponding number of columns of detectors is 336 columns. The collimator aperture size is 256*0.625, and the corresponding number of slices of detectors is 256 slices, where the unit of 0.625 is mm, it is the size of a single detector. Thus, an amount of the valid detectors participating in data acquisition is 336*256=86016. After analogue to digital conversion, data detected by one detector for one sampling may be represented as 16 bits, and the amount of acquisition data output by the valid detectors for one sampling is 16*86016=1376256 bits.

The data acquisition density may be referred as a number of sampling times per unit time. It should be noted that the data acquisition density is related to dedicated lesions, and different lesions have different acquisition densities.

For example, data acquisition density with a lesion in a heart is greater than data acquisition density with a lesion in a leg. Because the heart periodically beats, in order to accurately scan situations of the heart movement at different times, it is necessary to increase sampling frequency of the detectors. That is, the data acquisition density is increased.

After an amount of data output by the valid detectors for one sampling are determined by the scanning field of view and the collimator aperture size, the data acquisition amount per unit time could be calculated based on the data acquisition density. In the above example, the amount of output data for one sampling is 1376256 bits, and the data acquisition density is 8957.5 times per second. The data acquisition amount per unit time is 1376256*8957.5=12327813120 bits≈12 Gb. Therefore, a data acquisition rate is approximately 12 Gb/s.

Step S303: a compression strategy and a decompression strategy are determined according to a first ratio of the data acquisition amount per unit time to the upper limit of the data transmission bandwidth.

As shown in FIG. 3B, a detail process of step S303 including the followings.

Step S3031: a ratio range corresponding to the first ratio is determined from a preset index table.

Step S3032: a compression ratio is obtained corresponding to the determined ratio range.

Step S3033: the compression strategy and the decompression strategy are determined based on the compression ratio.

As shown in Table 1, the relationships among the first ratio, the ratio range, the compression ratio and the compression strategy are described.

TABLE 1

| First | Ratio | Compression | Index | Compression | Decompression |
|---|---|---|---|---|---|
| ratio | range | ratio | value | strategy | strategy |
| 0.5 | (0-1] | 1 | A1 = 1 | C(A1) | D(A1) |
| 1.1 | (1-1.2] | 1.2 | A2 = 2 | C(A2) | D(A2) |
| 1.3 | (1.2-1.4] | 1.4 | A3 = 3 | C(A3) | D(A3) |

A compression strategy may indicate a dedicated compression algorithm. Since the optional compression algorithm for CT data is limited, it is impossible for each of first ratio corresponding to different compression strategies. Instead, first ratios within a ratio range may adopt one compression strategy, and a compression strategy corresponds to a compression ratio. In order to successfully transmit data, the compression ratio may be equal to the maximum value in the corresponding ratio range. For example, the compression ratio for the ratio range 1.2-1.4 is 1.4. When the first ratio is 1.3, a compression strategy with a compression ratio of 1.4 is adopted.

A1, A2, and A3 represent index values, and the index value refers to an index of the ratio range. C represents the compression strategy and has a one-to-one correspondence with respect to the index value, that is, one-to-one correspondence with respect to the compression ratio. D represents the decompression strategy and also has a one-to-one correspondence with respect to the index value, that is, one-to-one correspondence with respect to the compression ratio. The compression strategy has a dedicated corresponding decompression strategy.

In addition, as shown in Table 1, when the first ratio is 0.5, since it is less than 1, the data can be successfully transmitted without taking compression. Therefore, the compression strategy C (A1) is not compressed.

It should be noted that the index value is proportional to the compression ratio in the example. Thus, the larger the index value, the larger the first ratio. In order to transmit the acquisition data through a data transmission system of a CT machine, the corresponding compression ratio should be a larger one.

The CT data processing method provided by the example determines a data acquisition amount per unit time when scanning a lesion according to a scanning field of view of the CT machine, a collimator aperture size and data acquisition density. Further, a corresponding first ratio is calculated. An index value is determined according to the first ratio, and a corresponding compression strategy is selected. Different first ratios correspond to different compression strategies, rather than a single compression strategy with a high compression ratio to meet transmission requirements for all scenarios of acquisition data in the prior art. In this example, when the first ratio is less than or equal to a certain preset value, such as 1, acquisition data may be transmitted without compression through the data transmission system. Thereby ensuring that data accuracy is not lost. When the first ratio is greater than 1, the corresponding compression strategy is adopted to ensure that the compressed data may be fully utilized the transmission bandwidth of the transmission system during transmission, thereby reducing the data accuracy loss of the acquisition data.

### Third Example

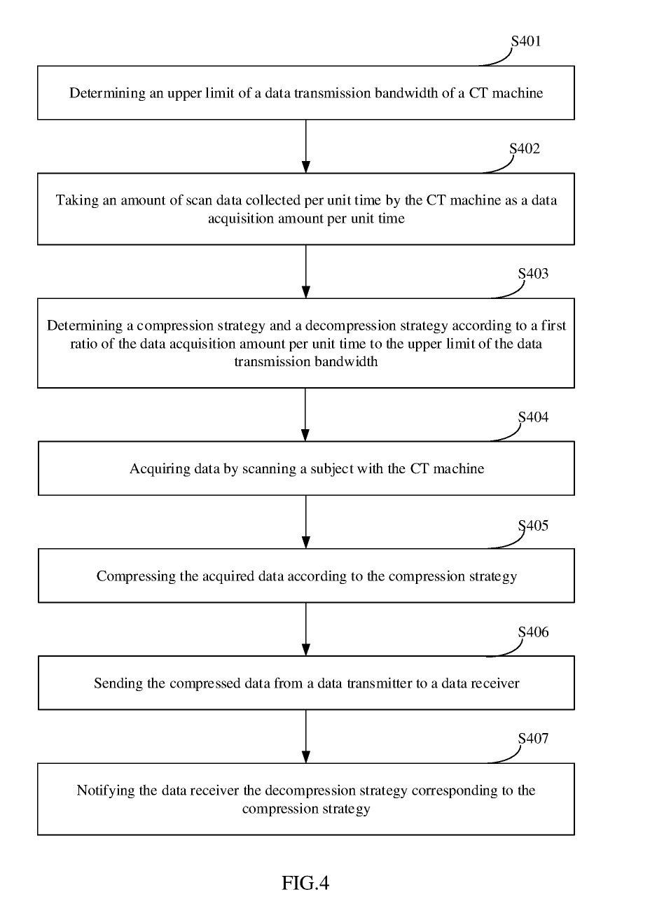

FIG. 4 is a flow chart illustrating a process of processing CT data according to the third example of the present disclosure. The CT data processing method provided in the example includes the following steps.

The process of steps S401-S403 may be similar with that of steps S301-S303 in the second example, which will not be repeated herein.

The process of steps S401-S403 may also be similar with that of steps S201-S203 in the first example, which will not be repeated herein.

Step S404: data by scanning a subject with the CT machine is acquired.

Step S405: the acquired data is compressed according to the compression strategy, where an amount of the compressed data per unit time is less than or equal to the upper limit of the data transmission bandwidth.

It should be noted that, after the scan data is compressed, the amount of the compressed data per unit time is less than or equal to the upper limit of the data transmission bandwidth. In this way, the compressed data may be transmitted by the data transmission system on time. If the amount of the compressed data per unit time is greater than the upper limit of the data transmission bandwidth, the compressed data may be overflowed.

The selected compression strategy should meet a requirement that the amount of the compressed data per unit time is less than or equal to the upper limit of the data transmission bandwidth.

For example, if the data acquisition amount per second is 12 Gb and the upper limit of the data transmission bandwidth is 10 Gbps, the first ratio is 1.2. A corresponding compression ratio may be 1.2, and a compression strategy may be lossless compression. Then the amount of the compressed data per second is 10 Gb, which is equal to the upper limit of the data transmission bandwidth.

For another example, if the data acquisition amount per second is 11 Gb and the upper limit of the data transmission bandwidth is 10 Gbps, the first ratio is 1.1 which is in a range from 1.0 to 1.2. A corresponding compression ratio may be 1.2, and a compression strategy may be lossless compression. Then the amount of the compressed data per second is 9.17 Gb, which is less than the upper limit of the data transmission bandwidth.

Step S406: the compressed data is sent from a data transmitter to a data receiver, where the data transmitter locates in a scanner of the CT machine, and the data receiver locates in a computer system of the CT machine.

Step S407 may be further performed after step S406.

S407: the data receiver is notified the decompression strategy corresponding to the compression strategy.

The decompression strategy corresponding to the compression strategy is sent to the data receiver, so that the data receiver may decompress received compressed data. Then the computer system may perform image reconstruction based on the decompressed data.

It should be noted that the decompression strategy may be sent to the data receiver together with the compressed data, or may be sent to the data receiver after finishing the compressed data transmission, or may be sent to the data receiver by other means. The present disclosure does not limit manners of sending the decompression strategy to the data receiver. The present disclosure does not limit the execution order for step S406 and step S407.

The data receiver locates in the computer system. The data receiver may perform decompression processing according to the decompression strategy, and then send decompressed data to the computer system for storage. Or the data receiver may send the compressed data to the computer system for storage directly, and the compressed data is decompressed by the computer system according to the decompression strategy.

In the CT data processing method provided by the example selects a corresponding compression strategy by using an upper limit of a data transmission bandwidth in the CT machine and an amount of scan data collected per unit time by the CT machine. Different compression strategies are adopted according to different first ratios. The data acquisition amount per unit time is different, and the corresponding compression strategy is also different, rather than a single compression strategy with a high compression ratio to meet transmission requirements for all scenarios of acquisition data in the prior art. In this example, when the acquisition data amount per unit time is small, a compression strategy with a high compression ratio may be avoid, that is, large data accuracy loss may be avoid. In this example, a compression strategy is selected to ensure that the amount of the compressed data per unit time is less than or equal to the upper limit of the data transmission bandwidth. In this way, the compressed data may be transmitted by the data transmission system on time. Therefore, the method provided by the present disclosure may improve the data accuracy as much as possible and fully utilize the bandwidth of a data transmission system.

Based on CT data processing methods provided by the above examples, a device for CT data processing is also provided by the present disclosure, which will be described below in detail in conjunction with accompanying drawings.

FIG. 5 is a schematic diagram illustrating a hardware structure of a CT data processing device according to an example of the present disclosure. The device includes a processor 510 and machine readable storage medium 520, where the processor 510 and the machine readable storage medium 520 are usually connected with each other via an internal bus 530. In other possible implementations, the device may also include a communication interface 540 to communicate with other devices or components.

In different examples, the machine readable storage medium 520 may be a Radom Access Memory (RAM), a volatile memory, a non-volatile memory, a flash memory, a storage drive (e.g., hard disk drive), a solid state hard disk, any type of storage disk (e.g., compact disk, Digital Video Disk (DVD)), or a similar storage medium, or a combination thereof.

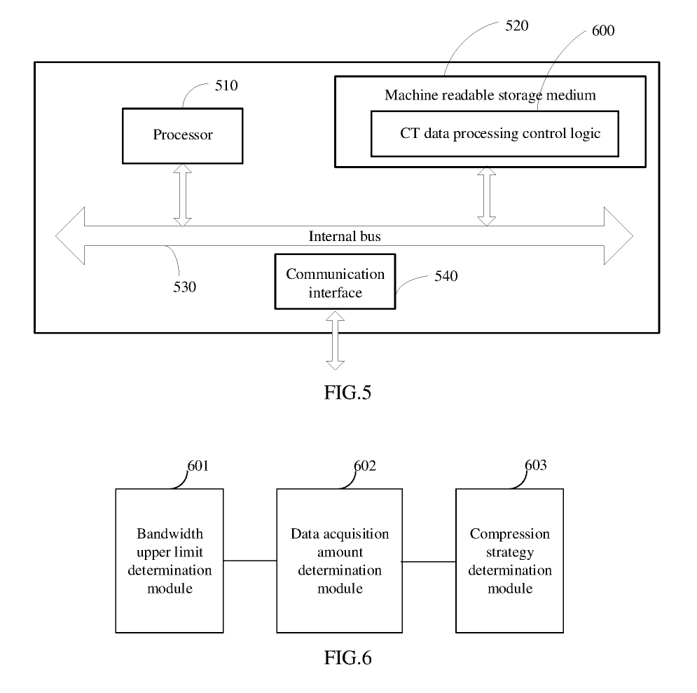

Further, control logic 600 for CT data processing is stored in the machine readable storage medium 520. As shown in FIG. 6, functionally, the control logic includes a bandwidth upper limit determination module 601, a data acquisition amount determination module 602, and a compression strategy determination module 603.

The bandwidth upper limit determination module 601 is configured to determine an upper limit of a data transmission bandwidth of a CT machine

The data acquisition amount determination module 602 is configured to take an amount of scan data collected per unit time by the CT machine as a data acquisition amount per unit time.

The compression strategy determination module 603 is configured to determine a compression strategy and a decompression strategy according to a first ratio of the data acquisition amount per unit time to the upper limit of the data transmission bandwidth.

The CT data processing device provided in this example selects a corresponding compression strategy by using an upper limit of a data transmission bandwidth in the CT machine and an amount of scan data collected per unit time by the CT machine. Different compression strategies are adopted according to different first ratios. In the case that the upper limit of the data transmission bandwidth is determined, the data acquisition amount per unit time is different, and the corresponding compression strategy is also different. Rather than a single compression strategy with a fixed compression ratio in the prior art, the solution provided by the present disclosure may adopt a compression strategy with a large compression ratio for a large amount of data acquired per unit time, and a compression strategy with a small compression ratio or even no compression for a small amount of data acquired per unit time. The greater the compression ratio, the greater loss of data accuracy. Therefore, the device provided by the present disclosure may improve the data accuracy as much as possible while ensuring normal data transmission, and fully utilize the bandwidth of a data transmission system.

A software implementation is taken as an example below to further describe how a device for CT data processing executes the control logic 600. In the example, the control logic 600 of the present disclosure should be understood as computer instructions stored in the machine readable storage medium 520. When the processor 510 on the CT data processing device of the present disclosure executes the control logic 600, the processor 510 may perform the following operations by invoking the instructions corresponding to the control logic 600 stored on the machine readable storage medium 520.

An upper limit of a data transmission bandwidth of a CT machine is determined.

An amount of scan data collected per unit time by the CT machine is taken as a data acquisition amount per unit time.

A compression strategy and a decompression strategy are determined according to a first ratio of the data acquisition amount per unit time to the upper limit of the data transmission bandwidth.

When the processor determines the compression strategy and the decompression strategy by reading machine readable instructions corresponding to the control logic in a storage medium, the followings may be specifically included.

A ratio range is determined corresponding to the first ratio from a preset index table.

A compression ratio is obtained corresponding to the determined ratio range, where the compression ratio refers to a ratio of an amount of data before compression to an amount of data after compression.

The compression strategy and the decompression strategy are determined based on the compression ratio.

The processor may further perform the following operations by invoking the instructions corresponding to the control logic 600 stored on the machine readable storage medium.

Data is acquired by scanning a subject with the CT machine.

The acquired data is compressed according to the compression strategy, where an amount of the compressed data per unit time is less than or equal to the upper limit of the data transmission bandwidth.

The compressed data is send from a data transmitter to a data receiver, where the data transmitter locates in a scanner of the CT machine, and the data receiver locates in a computer system of the CT machine.

The processor may further perform the following operations by invoking the instructions corresponding to the control logic 600 stored on the machine readable storage medium.

The data receiver is notified of the decompression strategy.

Further, the upper limit of the data transmission bandwidth may be a smaller one between a transmission rate of a slip ring and a transmission rate of an optical fiber link in the CT machine.

Further, the data acquisition amount per unit time may be determined according to a scanning field of view of the CT machine, a collimator aperture size of the CT machine and data acquisition density.

Further, an example of the present disclosure also provides a computer readable storage medium that stores instructions. When the instructions are executed by one or more processors, the one or more processors are caused to perform a method of CT data processing.

An upper limit of a data transmission bandwidth of a CT machine is determined.

An amount of scan data collected per unit time by the CT machine is taken as a data acquisition amount per unit time.

A compression strategy and a decompression strategy are determined according to a first ratio of the data acquisition amount per unit time to the upper limit of the data transmission bandwidth.

For simplicity and illustrative purposes, the present disclosure is described by referring mainly to examples thereof. In the above descriptions, numerous specific details are set forth in order to provide a thorough understanding of the present disclosure. It will be readily apparent however, that the present disclosure may be practiced without limitation to these specific details. In other instances, some methods and structures have not been described in detail so as not to unnecessarily obscure the present disclosure. As used herein, the terms “a” and “an” are intended to denote at least one of a particular element, the term “includes” means includes but not limited to, the term “including” means including but not limited to, and the term “based on” means based at least in part on.

The above description is merely preferred examples of the present disclosure and is not intended to limit the present disclosure in any form. Although the present disclosure is disclosed by the above examples, the examples are not intended to limit the present disclosure. Those skilled in the art, without departing from the scope of the technical scheme of the present disclosure, may make a plurality of changes and modifications of the technical scheme of the present disclosure by the method and technical content disclosed above.

Therefore, without departing from the scope of the technical scheme of the present disclosure, based on technical essences of the present disclosure, any simple alterations, equal changes and modifications should fall within the protection scope of the technical scheme of the present disclosure. Accordingly, other embodiments are within the scope of the following claims.

What is claimed is:

1. A method of processing CT data, comprising:
determining an upper limit of a data transmission bandwidth of a CT machine;
taking an amount of scan data collected per unit time by the CT machine as a data acquisition amount per unit time; and
determining a compression strategy and a decompression strategy according to a first ratio of the data acquisition amount per unit time to the upper limit of the data transmission bandwidth.

2. The method of claim 1, wherein determining the compression strategy and the decompression strategy comprises:
determining a ratio range corresponding to the first ratio from a preset index table;
obtaining a compression ratio corresponding to the determined ratio range, wherein
the compression ratio refers to a ratio of an amount of data before compression to an amount of data after compression; and
determining the compression strategy and the decompression strategy based on the compression ratio.

3. The method of claim 1, further comprising:
acquiring data by scanning a subject with the CT machine;
compressing the acquired data according to the compression strategy, wherein
an amount of the compressed data per unit time is less than or equal to the upper limit of the data transmission bandwidth; and
sending the compressed data from a data transmitter to a data receiver, wherein
the data transmitter locates in a scanner of the CT machine, and
the data receiver locates in a computer system of the CT machine.

4. The method of claim 3, further comprising:
notifying the data receiver of the decompression strategy.

5. The method of claim 1, wherein the upper limit of the data transmission bandwidth is a smaller one between a transmission rate of a slip ring and a transmission rate of an optical fiber link in the CT machine.

6. The method of claim 1, wherein the data acquisition amount per unit time is determined according to a scanning field of view of the CT machine, a collimator aperture size of the CT machine and data acquisition density.

7. A CT data processing device, the device is applied to a CT system, the device comprising a processor, wherein by reading and executing machine executable instructions corresponding to a control logic for processing CT data and stored on a machine readable storage medium, the processor is caused to:
determine an upper limit of a data transmission bandwidth of a CT machine;
take an amount of scan data collected per unit time by the CT machine as a data acquisition amount per unit time; and
determine a compression strategy and a decompression strategy according to a first ratio of the data acquisition amount per unit time to the upper limit of the data transmission bandwidth.

8. The device of claim 7, wherein when determining the compression strategy and the decompression strategy, the machine executable instructions cause the processor to:
determine a ratio range corresponding to the first ratio from a preset index table;
obtain a compression ratio corresponding to the determined ratio range, wherein
the compression ratio refers to a ratio of an amount of data before compression to an amount of data after compression; and
determine the compression strategy and the decompression strategy based on the compression ratio.

9. The device of claim 7, wherein the machine executable instructions further cause the processor to:
acquire data by scanning a subject with the CT machine;
compress the acquired data according to the compression strategy, wherein
an amount of the compressed data per unit time is less than or equal to the upper limit of the data transmission bandwidth; and
send the compressed data from a data transmitter to a data receiver, wherein
the data transmitter is in a scanner of the CT machine, and
the data receiver is in a computer system of the CT machine.

10. The device of claim 9, wherein the machine executable instructions further cause the processor to:
notify the data receiver of the decompression strategy.

11. The device of claim 7, wherein the upper limit of the data transmission bandwidth is a smaller one between a transmission rate of a slip ring and a transmission rate of an optical fiber link in the CT machine.

12. The device of claim 7, wherein the data acquisition amount per unit time is determined according to a scanning field of view of the CT machine, a collimator aperture size of the CT machine, and data acquisition density.

13. A machine readable storage medium coupled to at least one processor and having machine-executable instructions stored thereon that, when executed by the at least one processor, causes the at least one processor to perform operations comprising:
determining an upper limit of a data transmission bandwidth of a CT machine;
taking an amount of scan data collected per unit time by the CT machine as a data acquisition amount per unit time; and
determining a compression strategy and a decompression strategy according to a first ratio of the data acquisition amount per unit time to the upper limit of the data transmission bandwidth.
